(19) United States
(12) Patent Application Publication
(10) Pub. No.: US 2024/0142920 A1
(43) Pub. Date: May 2, 2024

(54) COMPONENT MATCHING DECISION SUPPORT TOOL

(71) Applicant: AMGEN INC., Thousand Oaks, CA (US)

(72) Inventors: Hillary Doucette COSTA, Boston, MA (US); Levi DeLUKE, Somerville, MA (US); Luis E. MONTES, Guaynabo, PR (US)

(73) Assignee: AMGEN INC., Thousand Oaks, CA (US)

(21) Appl. No.: 18/550,645

(22) PCT Filed: Mar. 14, 2022
(86) PCT No.: PCT/US2022/020113

§ 371 Date: Sep. 14, 2023

Related U.S. Application Data
(60) Provisional application No. 63/161,620, filed on Mar. 16, 2021.
(60) Provisional application No. 63/178,643, filed on Apr. 23, 2021.

(51) Int. Cl. G05B 13/02; G16H 20/10

(52) U.S. Cl. CPC G05B 13/028; G16H 20/10

(57) ABSTRACT

A method of reducing combination device variability includes identifying potential combinations of at least first and second components, where each combination can form one or more units of the combination device. The method also includes, for each potential combination, predicting a property or result of the units of the combination device when formed from at least the first and second components of the combination, at least by applying values of one or more characteristics of the first component of the combination, and values of one or more characteristics of the second component of the combination, as inputs to a predictive model. The method also includes selecting, based on the predicted properties or results for the potential combinations, a subset of combinations from among the potential combinations, and providing an indication of the selected subset of combinations.

### FIELD OF THE DISCLOSURE

The present application generally relates to the assembly or manufacture of devices, and more specifically relates to techniques for selecting specific components or component batches/lots to be used when assembling or manufacturing devices.

### BACKGROUND

In many manufacturing/production contexts, different types of components are combined to form a combined device. In the pharmaceutical industry, for example, fluid drug products are often combined with (i.e., used to fill) syringes to produce fluid-filled syringes. As another example, fluid-filled syringes can be combined with (i.e., fitted with) autoinjector subassemblies to produce complete autoinjectors. As yet another example, lyophilized drug products can be combined with (i.e., used to fill) vials or other containers. In each of these and other scenarios, multiple batches of each component may be available (e.g., in inventory) to be combined with each other. Due to component variability (e.g., batch-level variability), the manner in which any two components are paired (or more generally, in which two or more components are matched) for a given unit of the combination device can influence the manner in which individual component tolerances “stack up” in the final product, leading to a wider distribution of possible values for characteristics of the combination device (e.g., values of functional outputs of the device, such as injection time for an autoinjector device). Even if tolerances of individual components are tightly controlled, characteristics of the final product may exhibit a relatively wide range of values, leading to higher reject rates, more user (e.g., customer or patient) complaints, and/or other issues. Moreover, making tolerances for the individual components tight enough to meet tolerance targets in the final product may be prohibitively expensive.

### BRIEF SUMMARY

To address some of the aforementioned drawbacks of current/conventional practices, embodiments described herein include systems and methods that use data associated with different components (e.g., batch-specific or unit-specific data for different components) to better pair or match those components (e.g., specific components or specific batches of components) when manufacturing or assembling a combination device. In this manner, variability of the combination device (e.g., around a nominal specification) can be reduced, thereby providing better consistency of product performance (e.g., fewer defects and/or fewer user complaints) as compared to conventional process control measures. The term “component” is used broadly herein to refer to any physical portion of a combination device (e.g., a structural subcomponent, a raw material, or a fluid or lyophilized drug or other substance that is used to fill another component that acts as a vessel, etc.), unless a more specific meaning is clearly indicated by the context of its use. Moreover, the term “combination device” is broadly used herein to refer to any device that is manufactured or assembled using two or more components, and may be a “final” product (e.g., for sale or distribution) or an intermediate stage of a product, unless a more specific meaning is clearly indicated by the context of its use.

In the techniques disclosed herein, a predictive machine learning model predicts at least one property or result of units of a combination device (which may or may not be a “final” product for sale or distribution) for each of a number of different combinations of components that may be used to form that combination device (e.g., for Batch 1 of Component A with Batch 1 of Component B, and for Batch 1 of Component A with Batch 2 of Component B, etc.). A predicted property may be a measure of device quality or performance (e.g., a standard deviation of a characteristic of the combination device across multiple manufactured units), for example. As another example, a predicted result may be an indication of whether user complaints (generally, or of a specific type) will occur, or how frequently such complaints will occur, etc. To make each of its predictions, the model operates on (i.e., accepts as inputs) values of one or more characteristics of each of the components being considered for combination. These model inputs may include measured values, identifiers, other predicted (or inferred) values, and/or other types of upstream manufacturing data associated with the components (e.g., associated with specific component batches). After the model predicts the property or result (or multiple properties and/or results) for each of the various component combinations, an optimizer (e.g., a linear optimizer) operates on the predicted values to determine which components (or component batches, etc.) should be matched to “best” (e.g., optimally) meet a desired measure of device quality or performance (e.g., a desired value of the property/properties and/or result/results that was/were predicted by the model).

Using the techniques disclosed herein, specifically for the purpose of pairing pre-filled syringe batches with autoinjector subassembly batches, it has been shown that the lot-to-lot variability of mean injection time can be reduced by 30% to 45% as compared to the conventional process of randomly pairing batches. Generally, as the number of available component batches/lots increases, and/or as the number of components required to manufacture/assemble a particular combination device increases, there can be a corresponding increase in the benefit of matching batches/lots of those components using the techniques disclosed herein. Moreover, a software tool implementing the techniques described herein can facilitate understanding, by human users, of why certain combinations of components or component batches are advantageous. For example, using such a tool, a user can develop a set of heuristics, or “rules of thumb,” to enable intuitive pairings of components to offset different sources of variability. Generally, the techniques described herein can help to reduce lot-to-lot variability in manufactured combination devices, and/or improve user (e.g., customer and/or patient) experience.

### BRIEF DESCRIPTION OF THE DRAWINGS

The skilled artisan will understand that the figures, described herein, are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that implements a component matching tool to match components in a manner that reduces variability across units of a manufactured combination device.

FIGS. 2A and 2B depict components of example combination devices for which the component matching tool of FIG. 1 may match specific components or specific component batches.

FIG. 2C depicts an example device/product genealogy.

FIG. 3A depicts an example process that may be implemented at least in part by the computing system of FIG. 1.

FIG. 3B depicts pairing decision stages in an example manufacturing process.

FIG. 4 depicts an example user interface that may be generated and/or populated by the component matching tool of FIG. 1.

FIG. 5 depicts a plot showing example predictions of mean injection time and mean activation force for potential syringe stopper, syringe barrel, and drug combinations.

FIG. 6 depicts a plot comparing predicted mean injection time for an autoinjector when randomly matching autoinjector components, when using heuristic rules to match autoinjector components, and when using a linear optimizer to match autoinjector components.

FIG. 7 depicts a plot of example SHAP and feature importance values for an implementation in which the combination device is an autoinjector.

FIG. 8 is a flow diagram of an example method for reducing combination device variability.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

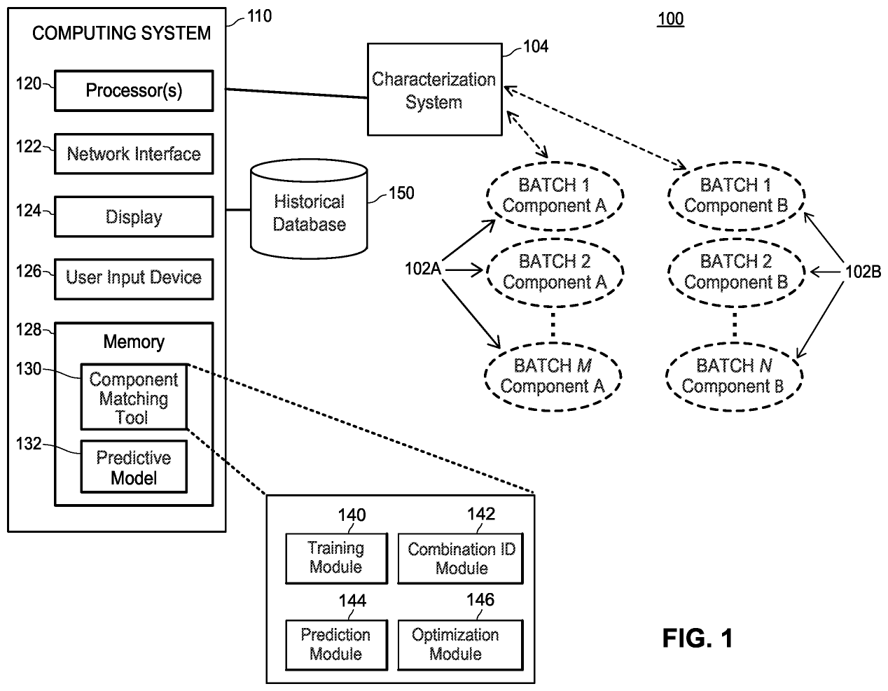

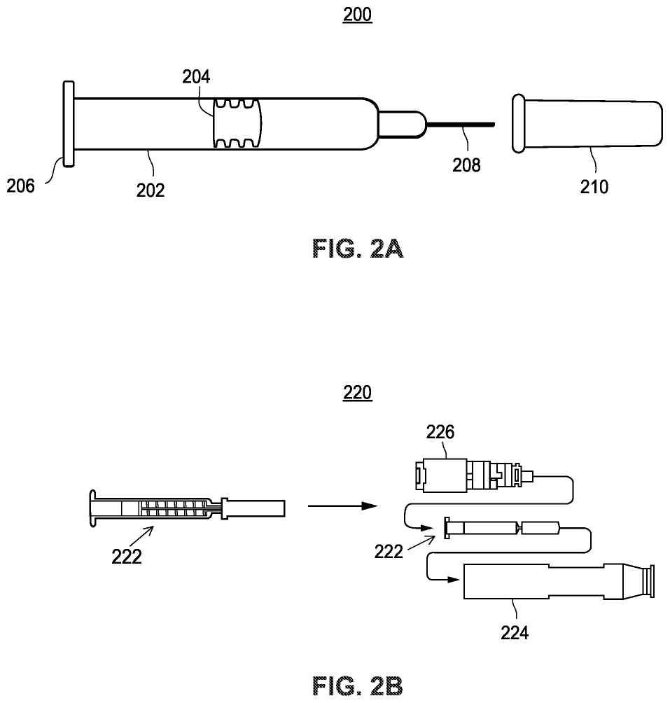

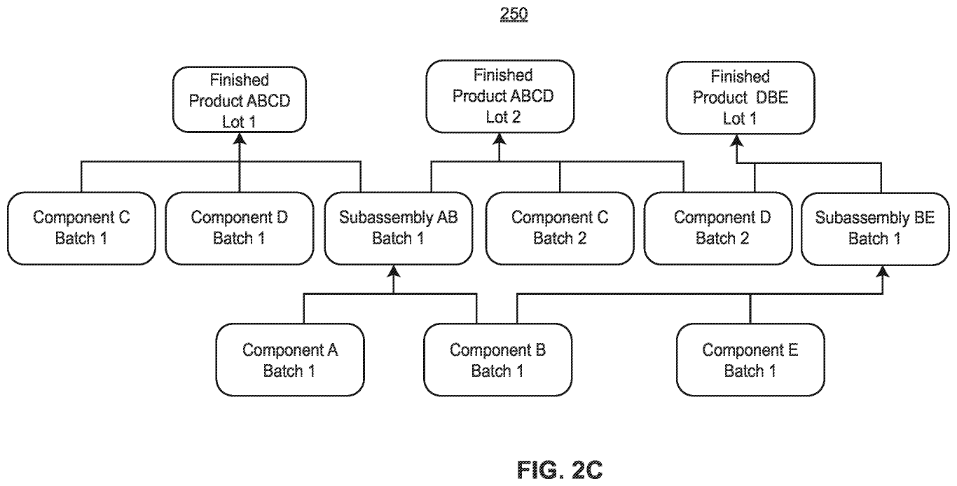

FIG. 1 is a simplified block diagram of an example system 100 that can implement the techniques disclosed herein. The example system 100 includes units of a Component A in M batches 102A and units of a Component B in N batches 102B (with M and N being integers that are the same or different, and with one or both of the numbers being greater than one). Component A and Component B may be any physical components that can be combined/assembled with each other to form a combination device. The combination device may be any device that is manufactured or assembled using two or more such components, and may be a “final” device/product (e.g., for sale or distribution) or an intermediate stage of a device/product (e.g., a subassembly of a piece of equipment). FIGS. 2A and 2B provide specific, non-limiting examples of Component A and/or Component B. For example, Component A may be the syringe 200 of FIG. 2A, which includes a barrel 202, a plunger/stopper 204, a flange 206, a needle 208, and possibly a needle shield 210, and Component B may be a fluid drug product that fills the syringe 200, such that the resulting combination device is a fluid-filled syringe. As another example, Component A may be a combination of the barrel 202 (integrated with flange 206) and the (pre-staked) needle 208 of FIG. 2A, and Component B may be the plunger/stopper 204 of FIG. 2A.

FIG. 2B depicts yet another example, corresponding to a subsequent manufacturing/assembly stage in which Component A is a fluid-filled syringe 222 and Component B includes both a forward autoinjector subassembly 224 and a rear autoinjector subassembly 226, with the combination device being a complete autoinjector device. Alternatively, the system 100 may treat the subassemblies 224 and 226 as separate components, and match batches of the fluid-filled syringe 222 (Component A), batches of the forward autoinjector subassembly 224 (Component B), and batches of the rear autoinjector subassembly 226 (Component C,. not shown in FIG. 1). It is understood that the techniques described herein may be applied to any number of components, and may be applied multiple times at successive manufacturing stages (e.g., to reduce the variability of fluid-filled syringes, and again at a subsequent manufacturing stage to reduce the variability of the final autoinjector device).

In some embodiments, the system 100 matches a set of multiple batches with another set of one or more batches. For example, batch 102A may correspond to a set of 10 batches of Component A, while batch 102B may correspond to one batch (or 10 batches, 20 batches, etc.) of Component B. While the descriptions herein primarily relate to matching different lots or batches of components (e.g., with each “batch” consisting of units manufactured in the same process run and/or same time window, etc. and at the same facility), in some embodiments the system 100 matches instead components on a unit-by-unit basis (e.g., if all units of each component are tested to provide measurement data that can be analyzed, rather than merely sampling a subset of batch units for measurement). Thus, it is understood that the embodiments described herein with respect to matching “batches” may instead match single units, match collections of units of arbitrary size (e.g., collections that do not correspond to “batches” as defined by a manufacturing process), or match sets of multiple batches, etc.

Moreover, while most embodiments discussed herein involve matching different types of components, in some embodiments the combination device is formed from at least two units of the exact same component, and batches 102A and 102B are subsets of all the batches of that component (i.e., Components A and B are the same component). Further, while most embodiments discussed herein involve pharmaceutical devices (e.g., autoinjectors), in other embodiments the combination device is a non-pharmaceutical device, such as a household appliance or an appliance subassembly, an automobile or automobile subassembly, medical equipment or a medical equipment subassembly, and so on. In an automotive embodiment, for example, Components A and B may be a cylinder head and a piston, respectively, of an engine.

Generally, the matching techniques described herein may be used to match components at any level of component genealogy (e.g., one or more hierarchical levels each containing one or more subassemblies or subcomponents, as well as the final assembly/product) within a manufacturing process, and may be applied at one or more levels of component genealogy. Certain conditions and/or qualities may make a given manufacturing process more amenable to the improvement through the techniques disclosed herein, including: (1) data (e.g., measurement data) from one or more components of the process is available before the corresponding manufacturing state(s) commence; (2) batch (or other grouping) genealogy data is available that links which components are used in a given final product or final product batch; (3) the available process/component data can predict the characteristic(s) of interest for the final product; and (4) the most predictive characteristics include characteristics of two or more components that will be combined (rather than just a single component where there is no opportunity to make any pairing decisions).

FIG. 2C depicts an example of one such device/product genealogy 250, in which different batches of different components are combined to create subassemblies and, ultimately, different lots of a final product. It is understood that, as the term “component” is used herein, all boxes beneath the “Finished Product” layer (e.g., “Component C,” “Subassembly AB,” etc.) can be viewed as combinable components. Thus, the genealogy 250 represents a manufacturing process in which a first level includes three distinct components, and the second level includes four distinct components (with two batches shown for “Component C” and two batches shown for “Component D”).

In some embodiments, different batches of one component (e.g., Component A or Component B) are of the same general component type, but are not precisely the same. Referring again to FIG. 1, for example, all batches 102A of Component A may be batches of the same syringe (e.g., the same part number or assembly of part numbers), while different batches 102B of Component B may be different fluid drug products. That is, the techniques described herein may be used to combine different batches of a single syringe type with different drug products. This can be advantageous because it has been found that, generally, the impact of component variability is not constant across different drugs due to the different drug properties (e.g., viscosity, protein concentration, etc.). Thus, optimal matching of drug types with different container batches, and/or of pre-filled syringes containing different drug types with different autoinjector subassembly batches, can reduce variability of the final autoinjector devices.

The system 100 includes a characterization system 104 that is generally configured to measure, and/or collect data representing, one or more characteristics of each batch 102A and each batch 102B. The characterization system 104 may include separate devices to measure characteristics of the two batches 102A, 102B (e.g., if Component A and Component B are sufficiently different in kind so as to require different measurement equipment, or if Component A and Component B are manufactured in different locations, etc.). If Component A is a syringe and Component B is a fluid drug product, for example, the characterization system 104 may include a first sensor (e.g., camera) and associated equipment (e.g., an optical comparator and a computing device that stores measurements) to automatically and/or manually measure inner barrel diameter of units of Component A, and a second sensor (e.g., viscometer) and associated equipment (e.g., a computing device that stores measurements) to automatically and/or manually measure drug product viscosity. The characterization system 104 may include, and/or collect data from, multiple data sources (e.g., different organizations, systems, etc.), and the data can correspond to one or more time periods.

In some embodiments, the characterization system 104 samples only a subset of units in each batch, and determines characteristic values that are representative of that batch (e.g., mean inner barrel diameter, mean viscosity, etc.). Generally, the characteristic values may be summary statistics from one or more tests and/or processes associated with each of the different components, such as mean, median, minimum, maximum, standard deviation, and specific quartile values.

In other embodiments (e.g., if components are matched on an individual basis rather than a batch-by-batch basis), the characterization system 104 measures every unit of Component A and every unit of Component B. In some embodiments, the characterization system 104 includes devices and associated equipment (e.g., sensor controllers, computing devices, etc.) to indirectly measure (i.e., “soft sense”) values of certain characteristics. For example, the characterization system 104 may include a Raman spectrometer that analyzes Raman scans of a fluid drug product to determine values indicative of chemical composition and molecular structure.

The system 100 also includes a computing system 110 coupled to the characterization system 104. The computing system 110 may include a single computing device, or multiple computing devices (e.g., one or more servers and one or more client devices) that are either co-located or remote from each other. In the example embodiment shown in FIG. 1, the computing system 110 includes one or more processors 120, a network interface 122, a display 124, a user input device 126, and a memory 128. In some embodiments, the computing system 110 includes a portion of the characterization system 104.

Each of the processor(s) 120 may be a programmable microprocessor that executes software instructions stored in the memory 128 to execute some or all of the functions of the computing system 110 as described herein. Alternatively, one or more of the processor(s) 120 may be other types of processors (e.g., application-specific integrated circuits (ASICs), field-programmable gate arrays (FPGAs), etc.).

The network interface 122 may include any suitable hardware (e.g., front-end transmitter and receiver hardware), firmware, and/or software configured to use one or more communication protocols to communicate with external devices and/or systems (e.g., the characterization system 104, another computing system that receives indications of optimal combinations from the computing system 110, or a server that provides an interface between the computing system 110 and the characterization system 104, etc.). For example, the network interface 122 may be or include an Ethernet interface. While not shown in FIG. 1, the computing system 110 may communicate with the characterization system 104, and/or with any device(s) that provide an interface between the computing system 110 and the characterization system 104, via a single communication network, or via multiple communication networks of one or more types (e.g., one or more wired and/or wireless local area networks (LANs), and/or one or more wired and/or wireless wide area networks (WANs) such as the Internet or an intranet, etc.).

The display 124 may use any suitable display technology (e.g., LED, OLED, LCD, etc.) to present information to a user, and the user input device 126 may be a keyboard or other suitable input device. In some embodiments, the display 124 and the user input device 126 are integrated within a single device (e.g., a touchscreen display). Generally, the display 124 and the user input device 126 may combine to enable a user to view and/or interact with visual presentations (e.g., graphical user interfaces or displayed information) output by the computing system 110, e.g., for purposes such as notifying users of recommended component batch combinations.

The memory 128 may include one or more physical memory devices or units containing volatile and/or non-volatile memory, and may include memories located in different computing devices of the computing system 110. Any suitable memory type or types may be used, such as read-only memory (ROM), solid-state drives (SSDs), hard disk drives (HDDs), and so on. The memory 128 stores the instructions of one or more software applications, including a component matching tool 130. The component matching tool 130, when executed by some or all of the processor(s) 120, is generally configured to: (1) collect the component batch data generated by the characterization system 104; (2) determine which combinations of batches 102A and 102B should provide better properties and/or results for the units of the combination device (e.g., narrower distributions of a particular characteristic of the combination device, and/or fewer user complaints, etc.); and (3) provide an indication of those combinations to a user and/or another computing system or application. To this end, the component matching tool 130 includes a training module 140, a combination identification module 142, a prediction module 144, and an optimization module 146. The modules 140 through 146 may be distinct software components or modules of the component matching tool 130, or may simply represent functionality of the component matching tool 130 that is not necessarily divided among different components/modules. For example, in some embodiments, the prediction module 144 and the training module 140 are included in a single software module. Moreover, in some embodiments, the different modules 140 through 146 may be distributed among multiple copies of the component matching tool 130 (e.g., executing at different devices in the computing system 110), or among different types of applications stored and executed at one or more devices of the computing system 110. The operation of each of the modules 140 through 146 is described in further detail below, with reference to the operation of the system 100.

As will also be described in further detail below, the computing system 110 is configured to access a historical database 150 for training purposes. The historical database 150 may store parameters values associated with past characterization data collected by the characterization system 104, and/or collected by other, similar devices or systems. For example, the historical database 150 may store individual (or mean, average, etc.) viscosities and/or protein concentrations of fluid drug products, barrel diameters and/or glide forces of syringes, etc., and possibly also values of other relevant parameters (e.g., time). In some embodiments, the historical database 150 also, or instead, stores categorical values for particular components or component batches, such as data indicating whether a drug fluid batch was observed to contain particles over a threshold size (e.g., over 20 um). The historical database 150 may also store “label” information representing actual properties and/or results for units of the combination device formed from specific combinations of batches of different components. In some embodiments, each label is a measured, numerical value associated with the resulting lot of combination devices. For example, each label may be a mean injection time or a mean activation force for a batch of autoinjector devices formed using a particular combination of component batches, or may a value or set of values that define a probability distribution of injection times or activation forces for the batch of autoinjector devices, etc. Alternatively, or in addition, each label may be an indicator of whether a specific type of user complaint (e.g., patient complaints, customer complaints, etc.), or user complaints generally, exceeded some threshold number or frequency for the resulting combination device, or the precise number, frequency, and/or likelihood of such complaints, etc. The database 150 may be stored in a persistent memory of the memory 128, or in a different persistent memory of the computing system 110 or another device or system. In some embodiments, the computing system 110 accesses the database 150 via the Internet using the network interface 122.

The training module 140 of the component matching tool 130 uses the characteristic (e.g., measurement) data and associated labels stored in the historical database 150 to train a predictive model 132, which is then used by the prediction module 144 to make predictions for units of combination devices that would result from specific combinations of component batches. The predictive model 132 may be a decision tree ensemble model (e.g., a gradient boosted tree model), a neural network, a support vector machine (SVM) model, a decision tree model, or any other suitable type of model. As a more specific example, the predictive model 132 may be an XGBoost model, which has been found to deliver superior performance for the disclosed techniques, as compared to certain other predictive model types (e.g., linear regression, LASSO, and random forest models). The predictive model 132 may include a classifier that predicts a particular classification of the resulting combination device for a particular combination of component batches (e.g., “within specification tolerance” or “not within specification tolerance,” or “good user experience,” “moderate user experience,” and “bad user experience,” etc.), a model that predicts a specific numerical value or values (e.g., a mean injection time, a standard deviation of injection time, an inter-quartile range of injection times, etc.), or a model that predicts a full probability distribution (e.g., if the predictive model 132 includes a modular neural network (MNN)), for example. The predictive model 132 may be created using open-source Python libraries, for example.

In some embodiments, the predictive model 132 is re-trained/refined, or a new predictive model is trained, for specific use cases. For example, the predictive model 132 or a new model may be trained using historical data that is specific to the manufacturing site to be used, the revision/version or type of the drug product being manufactured, a particular time period, and so on. Moreover, in embodiments where one or more labels are representative of user experience, the labels may be restricted to particular, relevant geographies (e.g., worldwide, US, or non-US), age groups, end-use environments (e.g., sample devices, for-sale devices, or hospital use devices), and so on.

The generation of predictive model 132 (e.g., in part or entirely by the training module 140, possibly with some user input) may include, for example: (1) preprocessing data (preparing historical data to be used by the predictive model 132 by normalizing and scaling features, converting data types, and imputing missing data); (2) splitting the preprocessed data into training and test/validation sets (to avoid overfitting the data and to estimate the performance of the predictive model 132 on unseen data); (3) selecting the model type (determining which supervised machine learning algorithm to use for a given prediction); (4) selecting and cross-validating hyper-parameters (to determine the best parameters for tuning a given model for a specific data set); (5) performing model fitting (using the training data and tuning parameters to create a predictive model, with the ability to select the optimal loss function to be minimized); (6) evaluating the predictive model 132 (evaluating model performance by using the trained predictive model 132 to make predictions on the test set); and (7) determining feature importance (providing visual representations of how the trained predictive model 132 arrives at a predicted output value based on the input data). Tuning parameters for an XGBoost model may include, for example, maximum depth, number of estimators, minimum child weight, learning rate, column sample by tree, subsample, alpha, gamma, and lambda. Model performance may be evaluated, for example, using the coefficient of determination (R²) metric.

As discussed in further detail below, the combination identification module 142 is generally configured to identify particular combinations of component batches (e.g., different combinations each pairing one of batches 102A with one of batches 102B), for which the prediction module 144 will then predict one or more properties and/or results of the combination devices that would result from that combination. As is also discussed in further detail below, the optimization module 146 is generally configured to process the combination-specific predictions output by the prediction module 144, in order to generate a set of combinations to be used in manufacture/assembly, or a set of combination recommended for manufacture/assembly.

As noted above, the computing system 110 may include one device or multiple devices and, if multiple devices, may be co-located or remotely distributed (e.g., with Ethernet and/or Internet communication between the different devices). In one embodiment, for example, a first server of the computing system 110 (including module 140) trains the predictive model 132, a second server of the computing system 110 collects measurements and/or other data from the characterization system 104, and a third server of the computing system 110 (including modules 142, 144, 146) receives the measurements and/or other data from the second server and uses a copy of the trained predictive model 132 to generate predictions based on the received measurements and/or other data. As another example, the third server of the above example does not store a copy of the trained predictive model 132, and instead utilizes the predictive model 132 by providing the measurements to the second server (e.g., if the predictive model 132 is made available via a web services arrangement). As used herein, and unless the context of the usage of the term clearly indicates otherwise, terms such as “running,” “using,” “implementing,” etc., a model (e.g., the predictive model 132) are broadly used to encompass the alternatives of directly executing a locally stored model, or requesting that another device (e.g., a remote server) execute the model. It is understood that still other configurations and distributions of functionality, beyond those shown in FIG. 1 and/or described herein, are also possible and within the scope of the invention disclosed herein.

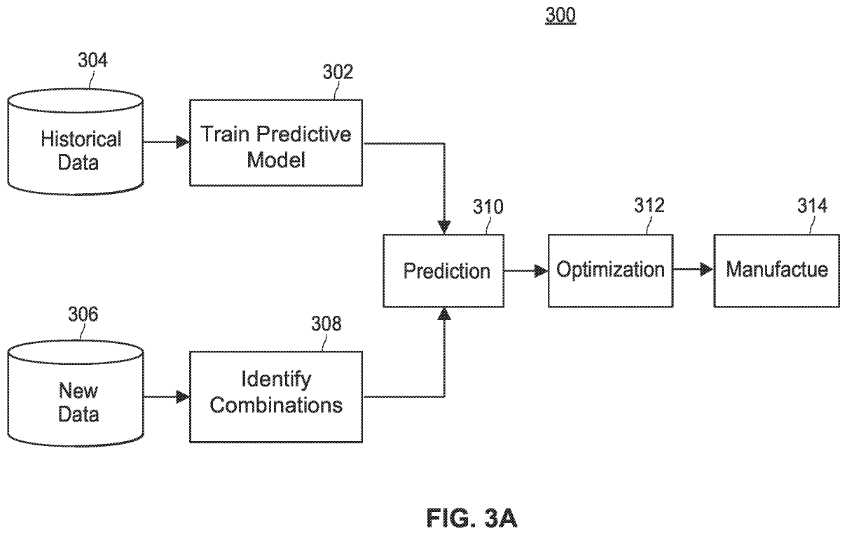

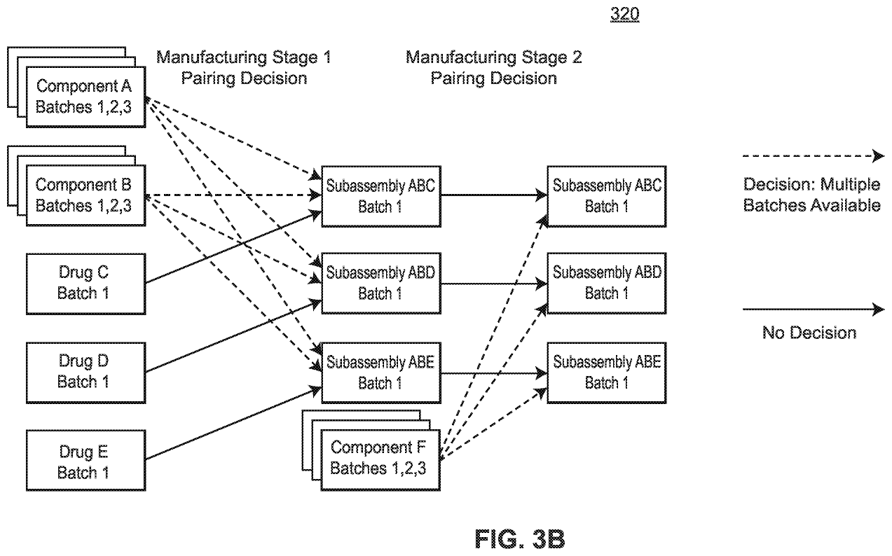

Operation of the system 100 will now be described in further detail, with reference to both the components of FIG. 1 and the process 300 depicted in FIG. 3A. In an initial training stage 302, the component matching tool 130 retrieves historical data 304 (e.g., past component measurements and associated labels) from the historical database 150, and the training module 140 trains the predictive model 132 using the retrieved historical data 304. For example, if Component A is a syringe and Component B is a drug product, the historical data 304 may include: (1) for each batch of syringes represented in the historical database 150, data indicative of glide force, shield removal force, barrel inner diameter, crush test results, trim max outer diameter of the plunger, max outer diameter of the plunger, and/or silicone weight of the plunger, e.g., a mean value for the batch; and (2) for each batch and/or type of drug represented in the historical database 150, a drug identifier and/or other information such as drug viscosity, protein concentration, manufacturing site, and/or density, e.g., with mean values where appropriate. As another example, if Component A is a pre-filled syringe and Component B includes a rear autoinjector subassembly, the historical data 304 may include: (1) for each batch of pre-filled syringes represented in the historical database 150, data indicative of extrusion force, breakloose force, protein concentration, particle characterization (e.g., whether particles above a certain size have been observed in the drug product, etc.), and/or any of the syringe-specific characteristics noted above (e.g., glide force, barrel inner diameter, etc.), e.g., a mean value for the batch; and (2) for each batch of rear autoinjector subassemblies represented in the historical database 150, a specification number, saline release testing results (e.g., injection time and/or activation force), and/or spring data (e.g., spring force), e.g., with mean values where appropriate.

The training module 140 trains the predictive model 132 to predict a specific property of the combination device, such as injection time or activation force (e.g., if the combination device is a pre-filled syringe or a complete autoinjector device). The value may be an expected mean value of the characteristic for all combination devices formed from the specific batches associated with a particular combination (e.g., Batch 1 of Component A with Batch 2 of Component B, etc.), for example, or an expected metric indicative of variability in the characteristics (e.g., a predicted standard deviation). In other embodiments, the training module 140 trains the predictive model 132 to predict a probability distribution for the combination device when formed from the particular combination of components.

In some embodiments, the predictive model 132 includes one or more preliminary model stages. For example, a first model stage of the predictive model 132 may transform inputs from the historical data 150 to values reflecting a reduced set of dimensions. As a more specific example, the predictive model 132 may apply a principal component analysis (PCA) technique to a set or subset of inputs to reduce that set or subset to n dimensions, and then apply the n values (one per dimension) as inputs to a subsequent model stage (e.g., a decision tree ensemble model or a neural network) that outputs predicted values. In such embodiments, the training module 140 may perform dimension reduction at stage 302 prior to applying inputs to the rest of the predictive model 132 for training purposes.

Stage 302 may also include validating and/or qualifying the trained predictive model 132 (e.g., using portions of the historical data 304 that were not used for training). Once satisfactorily trained and possibly validated/qualified, the prediction module 144 of the component matching tool 130 can use the predictive model 132 at stage 310 to predict values of one or more combination device characteristics for each combination of components or component batches being considered. Before stage 310, however, the combination identification module 142 of the component matching tool 130 determines which combinations of component batches (and thus, which subsets of new data 306) are to analyzed. The new data 306 includes data indicative of characteristics of component batches that are to be assembled to form new units of the combination device, and may include the same types of measurements and/or other component data described above with respect to the historical data 304 (e.g., syringe glide force, drug product identifier, etc.).

The combination identification module 142 may determine the combinations to be considered by, for example, receiving lists of batch identifiers for different components (e.g., Component A and Component B) that were manually entered by a user (e.g., via the user input device 126), or from another computing system, storage device, or application, and then automatically generating each possible permutation based on the batch identifier lists. In other embodiments, the combination identification module 142 at stage 308 also filters out (omits) combinations that are not feasible or practical (e.g., where due to timing/sequencing of the manufacturing process, two batches could not possibly be combined), to avoid wasting processing resources at stage 310. In other embodiments, the combination identification module 142 identifies/determines the appropriate component/batch combinations to be considered merely by receiving an indication of those combinations (e.g., as manually entered by a user, or from another computing system, storage device, or application). In some embodiments and/or scenarios, the outputs of (and/or inputs to) the combination identification module 142 are limited on a time basis, e.g., such that component batches can only be set aside in a “holding” area for a limited time, in order to avoid large disruptions to the manufacturing process.

At stage 310, the prediction module 144 applies the portions/subsets of the new data 306 that correspond to the components/batches of the combinations identified at block 308 as inputs to the predictive model 132. The prediction module 144 may operate on the data 306 for each identified combination sequentially, i.e., by predicting the property or result of interest for one combination before proceeding to the next combination. Alternatively (e.g., if the processor(s) 120 can implement multiple instances of the prediction module 144), the component matching tool 130 may make predictions for multiple combinations in parallel. As noted above, the predictive model 132 can include a dimension reduction stage, in some embodiments, in which case stage 310 includes reducing the data dimensionality prior to inputting the reduced-dimension data to the predictive model 132.

The output at stage 310 includes a prediction of one or more properties and/or results for each combination that was identified at stage 308 (i.e., the same types of properties and/or results represented by the labels used at the training stage 302). At stage 312, the optimization module 146 applies the predicted propert(ies) and/or result(s) for all of the identified combinations as inputs to an optimizer at stage 312, in order to determine which set of combinations provides the “best” (e.g., optimal) performance. The optimizer may be a linear optimizer. For example, the optimization module 146 may use PuLP (a Python linear programming application programming interface (API)) to define an objective function and invoke external solvers.

In some embodiments where the prediction module 144 predicts only a single property (e.g., a standard deviation of injection time across all combination device units resulting from a particular component combination), the optimization module 146 solves the following objective function:

$$\\ min ( {\sum }_{i = 1}^{i = n} {( {J}_{i} - {J}_{mean} )}^{2} {X}_{i} )$$

where Jᵢ is the predicted mean combination device property (e.g., mean injection time) for the i-th combination, Jₘₑₐₙ is the actual (e.g., measured or known) mean combination device property when using a given drug, and Xᵢ is either zero or one depending on the permutation being considered and the value of i. The integer n represents the total number of permutations of the combinations identified at stage 308. For example, if 12 batches of Component A and 20 batches of Component B are available, and if the combination identification module 142 does not rule out any combinations, then n=12×20=240. When minimizing the objective function above, the optimization module 146 sets the values of Xᵢ such that no two mutually exclusive combinations are selected for any given sum. As a relatively simple example, if two batches of Component A and three batches of Component B are available, the optimization module 146 may compute the sum portion of the above objective function by setting Xᵢ as follows:

| Combination | Comp A | Comp B | | | | | | |
|---|---|---|---|---|---|---|---|---|
| number (i) | Batch | Batch | X₁ | X₂ | X₃ | X₄ | X₅ | X₆ |
| 1 | A1 | B1 | 1 | 1 | 0 | 0 | 0 | 0 |
| 2 | A1 | B2 | 0 | 0 | 1 | 1 | 0 | 0 |
| 3 | A1 | B3 | 0 | 0 | 0 | 0 | 1 | 1 |
| 4 | A2 | B1 | 0 | 0 | 1 | 0 | 1 | 0 |
| 5 | A2 | B2 | 1 | 0 | 0 | 1 | 0 | 1 |
| 6 | A2 | B3 | 0 | 1 | 0 | 0 | 0 | 0 |

To minimize the objective function in this example, the optimization module 146 would determine which of the columns X₁ through X₆ results in the lowest sum, and select that as the desired set of combinations.

In some embodiments, the optimization module 146 minimizes an objective function that includes multiple terms each similar to the term shown in the function above, e.g., with one term for each property or result that is predicted by the prediction module 144. For example, the objective function may include a first term corresponding to mean injection time and a second term corresponding to mean number of complaints (e.g., 0.1 if one complaint per 10 units is predicted). Some or all of the individual terms of the objective function may be weighted depending on the perceived importance of each term to the overall performance of the combination device.

The optimization module 146 provides an output indicating the set/permutation of combinations that minimized the objective function, and at stage 314 lots of the combination device are manufactured using the indicated combinations (e.g., using commercial-scale production equipment). In some embodiments, the combination matching tool 130 causes a display (e.g., the display 124) to present a user interface that shows the resulting combinations to a user, and the user may then (possibly after a manual review/confirmation) take one or more actions to ensure that the manufacturing at stage 314 uses those combinations. In other embodiments, the combination matching tool 130 sends data indicative of the resulting combinations to another computing system or application, which in turn automatically causes the manufacturing process to use those combinations (e.g., by controlling the appropriate conveyance/routing equipment).

In some embodiments, measurements of the combination devices manufactured at stage 314 are manually or automatically obtained, and used as labels to compare against the predictions that were made at stage 310. For each of one or more batch combinations, for example, the corresponding input data (from the new data 306) that was applied to the predictive model 132, along with the corresponding label, may be fed back to the training module 140, and the training module 140 may use the data and label to refine/update the predictive model 132 through further training.

FIG. 3B depicts pairing decision stages in an example manufacturing process 320. At a first stage, different drugs (and possibly different batches of each drug) are paired to other components (and possibly different batches of those other components). For example, different fluid drug products may be paired with different types of assembled syringes, and/or different batches of those syringes. The second stage depicted in FIG. 3B may represent the pairing of particular filled syringes with particular autoinjector subassemblies, for example. The component matching tool 130 may match components and/or batches (as shown by the dashed lines in FIG. 3B) in an attempt to optimize one or more characteristic values, such minimizing probability of glass breakage, minimizing a standard deviation from a desired injection time, and so on. Typically, there is greater data availability and predictive power as the stage number increases (i.e., as one draws closer to the final product), but less of an opportunity for improvement through the matching decision.

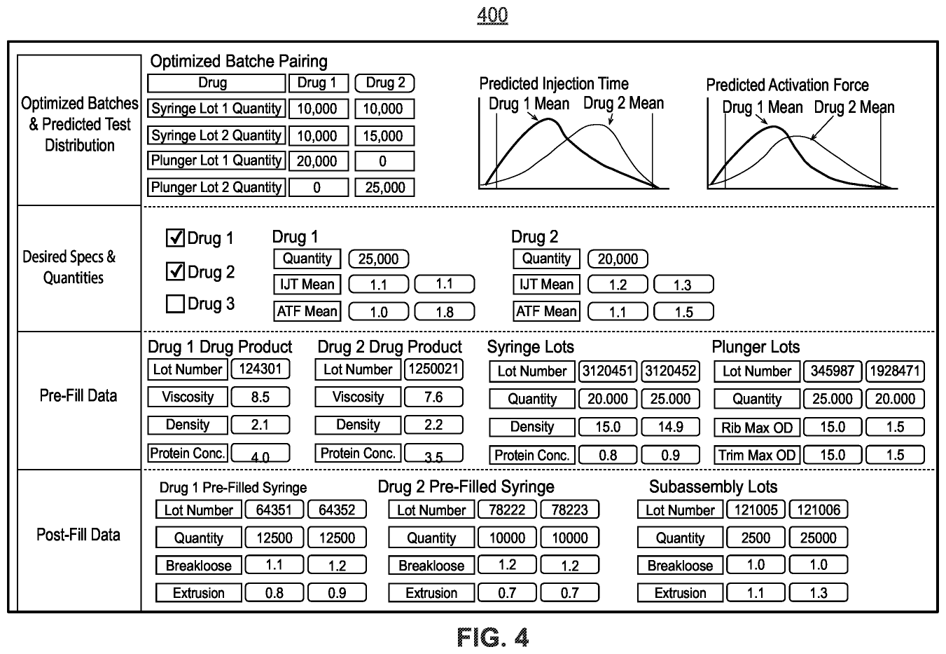

FIG. 4 depicts an example user interface 400 that may be generated and/or populated by the component matching tool 130 of FIG. 1 or a similar application, in some embodiments. FIG. 4 represents an embodiment in which component matching is performed at two successive stages: the pre-fill stage in which batches/lots of empty syringes are matched to different drug products (“Drug 1,” “Drug 2,” and “Drug 3”), and the post-fill stage in which those filled (“pre-filled”) syringes are matched to batches/lots of autoinjector subassemblies. The quantities shown in FIG. 4 are purely for illustration purposes, and are not necessarily realistic values. Moreover, it is understood that the values may be associated with any suitable units (e.g., Newtons for mean activation force and syringe glide force, seconds or milliseconds for mean injection time, etc.).

In the row of the user interface 400 labeled “Desired Specs & Quantities,” a user can select (e.g., via the user input device 126) particular drugs to be considered (in the example shown, “Drug 1” and “Drug 2”). In the row labeled “Pre-Fill Data,” the user can enter (e.g., via the user input device 126) and/or view (e.g., via the display 124) values of characteristics associated with particular lots/batches of the selected drugs, and of characteristics associated with particular lots/batches of syringes that can be used to hold those drugs. In the example shown, values of characteristics of plunger lots/batches are also displayed, and the component matching tool 130 matches batches of the drug, the syringe, and the plunger. With reference to FIG. 1, for example, the drug may be Component A and the syringe may be Component B, while the plunger may be a Component C not shown in FIG. 1. In the row labeled “Post-Fill Data,” the user can enter (e.g., via the user input device 126) and/or view (e.g., via the display 124) values of characteristics associated with particular lots/batches of pre-filled syringes (on a per-drug basis), and of characteristics associated with particular lots/batches of autoinjector subassemblies.

In the example user interface 400, results for each of the selected drugs are shown in the top two rows. In particular, the top row shows optimized batch pairing of drugs with syringes and plungers, as well as plots of predicted injection time and activation force (per selected drug), and the second row shows values of the predicted mean injection time and mean activation force per selected drug.

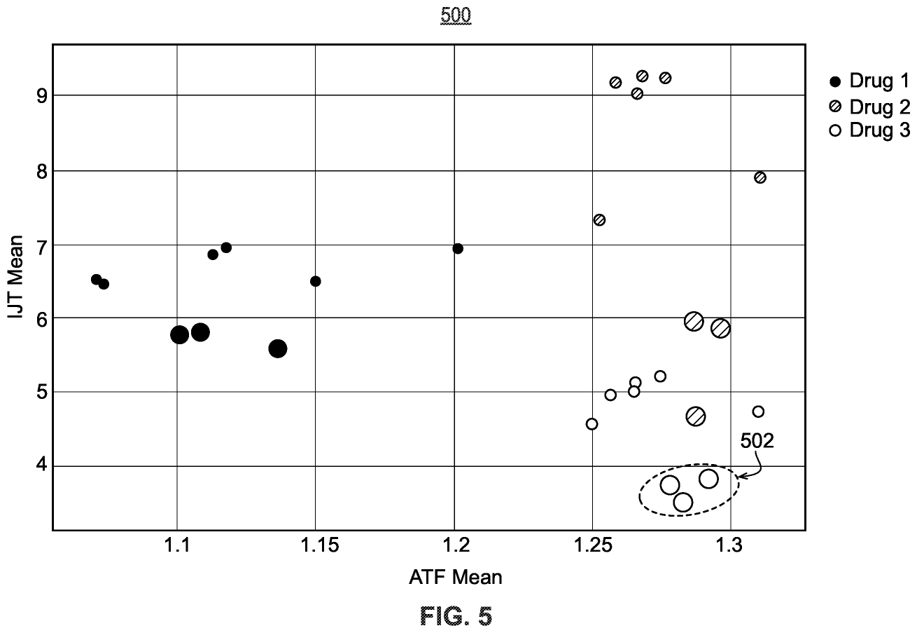

FIG. 5 depicts a plot 500 showing example predictions of mean injection time and mean activation force for potential syringe stopper, syringe barrel, and drug combinations. In some embodiments, the combination matching tool 130 generates and/or populates a visualization that includes a plot similar to the plot 500, e.g., to help users understand how different drugs impact the variability of component batches. In FIG. 5, each circle represents a different combination of syringe stopper/barrel batch and drug type, with enlarged circles representing combinations that use one particular batch/lot of syringe barrels. As seen from FIG. 5, filling syringe barrels from this particular batch with the drug represented by Drug 3 (at location 502) results in the lowest injection time.

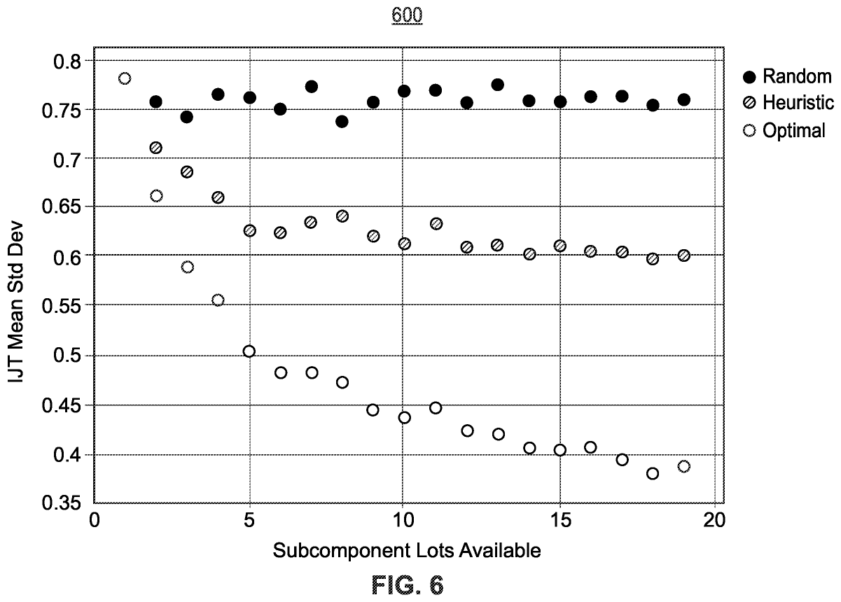

FIG. 6 depicts a plot 600 comparing predicted standard deviation of mean injection time for an autoinjector when randomly matching autoinjector components (i.e., similar to the conventional approach), when using heuristic rules to match autoinjector components (i.e., heuristic rules that attempt to offset injection time contributions of the pre-filled syringe and the autoinjector subassembly), and when using a linear optimizer to match autoinjector components (i.e., a tool similar to component matching tool 130). In particular, as seen in FIG. 6, simulation shows a 30-45% reduction in lot-to-lot variation in average injection time when optimally pairing pre-filled syringes with autoinjector subassemblies.

The above discussion assumes that it is already known which characteristic values should be used to determine component matching (i.e., the characteristics for which values should be measured/collected for use as inputs to the predictive model 132). However, understanding which characteristic values (and which components at various genealogical levels) are most strongly correlated with final device/product characteristics can be challenging. Moreover, such correlations may vary over time, manufacturing site, product revisions, and so forth. Thus, there is a need for a generalized tool that quickly identifies correlations across cross sections of available data.

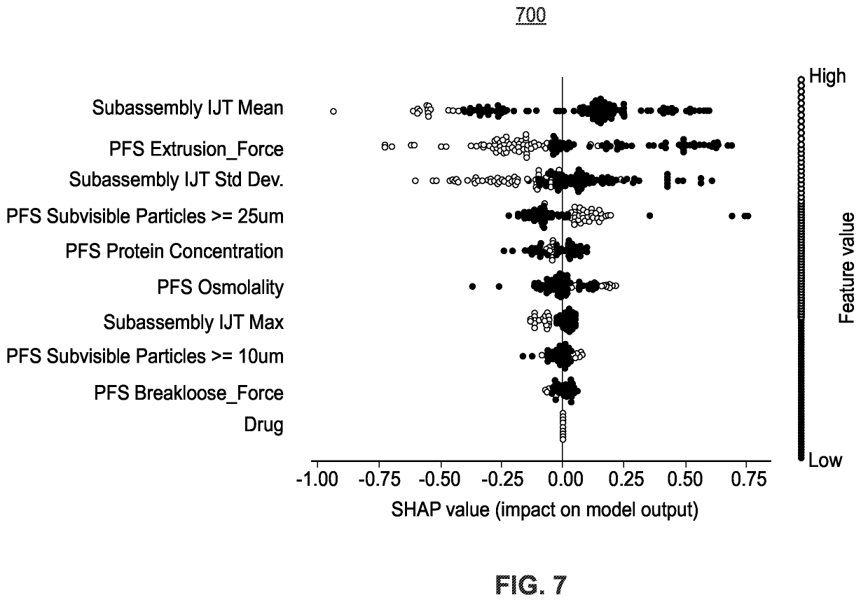

To this end, the component matching tool 130 (or another software tool) may generate and/or populate a visualization such as that shown as plot 700 in FIG. 7. The plot 700 represents example SHAP (Shapley additive explanations) and feature importance values for an implementation in which the combination device is an autoinjector. The plot 700 can help users understand the significance of certain component characteristics and how they may be used, for example, to counteract each other. For example, the heavy concentration of positive SHAP values for the subassembly mean injection time and the heavy concentration of negative SHAP values for the PFS (pre-filled syringe) extrusion force suggest that the two characteristics may be particularly useful in offsetting each other to achieve lower variability in the final autoinjector devices. A user may be able to apply such information to create or refine heuristic rules for matching batches of autoinjector components, for example, or to identify areas upon which to focus (e.g., areas to tighten specification tolerances) when manufacturing components of the autoinjector. The component matching tool 130 (or another software tool) may also, or instead, generate and/or populate one or more other types of visualizations, such as a correlation heat map or a pair plot. For example, a correlation heat map may visually depict (e.g., with color coding) the correlations between assembly date, an average customer experience metric, one or more process parameters, one or more statistics (e.g., mean and standard deviation) for one or more final lot parameters, one or more statistics (e.g., mean and standard deviation) for one or more component parameters, and so on.

In some embodiments, the following process is implemented (by the component matching tool 130 or other software) to create the predictive model 132: (1) the predictive model 132 is initially trained and evaluated using the original input set of data features (characteristics); (2) the features are ordered by importance as calculated using SNAP; (3) the least important input data feature are removed from the included features; (4) the predictive model 132 is retrained with the new, smaller set of data features/characteristics; (5) steps 2-4 are repeated until the input data features list reaches a desired length; and (6) the entire process is (optionally) repeated to determine differences between model performance and selected features/characteristics between runs causes by random seeding of the training and test sets.

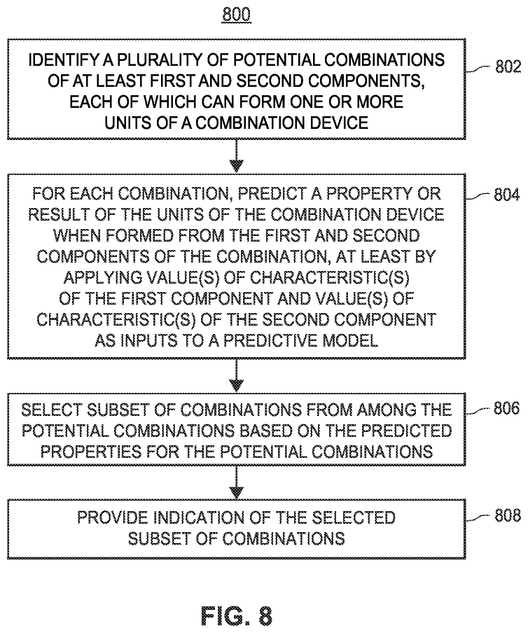

FIG. 8 is a flow diagram of an example method 800 for reducing combination device variability. The method 800 may be implemented in whole or in part by a computing system (e.g., computing device or devices), such as the computing system 110 of FIG. 1 (e.g., by the processor(s) 120 executing instructions of the component matching tool 130), for example.

At block 802, potential combinations of first components and second components (e.g., Components A and B of FIG. 1) are identified. Each of the potential combinations can form one or more units of a combination device (e.g., an autoinjector). For example, each potential combination may be a combination of a particular batch of the first components and a particular batch of the second components. Block 802 may include receiving a first set of identifiers of different batches of the first component and a second set of identifiers of different batches of the second component, and forming different pairs/permutations each consisting of an identifier of the first set and an identifier of the second set, or may simply include receiving an indication of each of the potential combinations. In some embodiments, block 802 includes selectively omitting infeasible combinations of components.

At block 804, for each combination of the potential combinations identified at block 802, a property or result of the units of the combination device (when formed from the first and second components of that combination) is predicted. Block 804 includes applying values of one or more characteristics of the first component of the combination, and values of one or more characteristics of the second component of the combination, as inputs to a predictive model (e.g., the predictive model 132). In embodiments where the potential combinations are combination of batches (i.e., sets of two or more) of the first and second components, the characteristics may be characteristics that are statistically representative of the respective batches (e.g., mean barrel inner diameter, etc.). Block 804 may include predicting a numerical value (e.g., a mean value or a standard deviation), a category (e.g., “frequent complaints likely” or “frequent complaints not likely”), or a probability distribution for a particular characteristics, for example.

At block 806, a subset of combinations, from among the potential combinations identified at block 802, is selected based on the predicted properties or results for the potential combinations (i.e., based on the properties or results predicted at block 804). Block 806 may include solving an objective function using the predicted properties or results as inputs to the objective function. In some embodiments, block 806 includes selecting the subset based on both one or more properties (e.g., injection time standard deviation) and one or more results (e.g., presence, amount, frequency, or likelihood of user complaints).

At block 808, an indication of the selected subset of combinations is provided. For example, block 808 may include causing a display (e.g., the display 124) to indicate the selected subset of combinations to a user, and/or may include sending data indicating the selected subset of combinations to a computing system or application.

In some embodiments, the method 800 also includes one or more additional blocks not shown in FIG. 8. For example, the method 800 may further include, for each combination of the potential combinations, predicting one or more additional properties or results of the units of the combination device (when formed from the first and second components of the combination), using one or more additional predictive models. In these embodiments, the objective function may include a plurality of function terms each corresponding to a different property or result of the combination device.

As another example, the method 800 may include an additional block, after block 808, in which manufacturing equipment (e.g., commercial-scale production equipment) assembles a plurality of units of the combination device in accordance with the subset of combinations that was selected at block 806 and indicated at block 808.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “connect,” “connected,” and “connection” refer to (and connections depicted in the drawings represent) an operational coupling or linking. Connected components can be directly or indirectly coupled to one another, for example, through another set of components.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A method of reducing combination device variability, the method comprising:
identifying, by one or more processors, a plurality of potential combinations of at least first components and second components, wherein each combination of the potential combinations can form one or more units of a combination device;
for each combination of the potential combinations, predicting, by the one or more processors, a property or result of the units of the combination device when formed from at least the first and second components of the combination, at least by applying (i) values of one or more characteristics of the first component of the combination, and (ii) values of one or more characteristics of the second component of the combination, as inputs to a predictive model;
selecting, by the one or more processors and based on the predicted properties or results for the potential combinations, a subset of combinations from among the potential combinations; and
providing, by the one or more processors, an indication of the selected subset of combinations.

2. The method of claim 1, wherein:
each combination of the potential combinations is a different pair consisting of (i) a batch of the first components and (ii) a batch of the second components; and
for each combination of the potential combinations,
the respective values of the one or more characteristics of the first component are statistically representative of the respective batch of the first components, and
the respective values of the one or more characteristics of the second component are statistically representative of the respective batch of the second components.

3. The method of claim 2, wherein identifying the plurality of potential combinations includes:
receiving a first set of identifiers of different batches of the first component;
receiving a second set of identifiers of different batches of the second component; and
identifying the plurality of potential combinations by forming different pairs each consisting of an identifier of the first set and an identifier of the second set.

4. The method of claim 1, wherein identifying the plurality of potential combinations includes at least one of omitting infeasible combinations, or receiving an indication of each of the plurality of potential combinations.

5. (canceled)

6. The method of claim 1, wherein selecting the subset of combinations includes solving an objective function using the predicted properties or results for the potential combinations as inputs to the objective function.

7. The method of claim 6, wherein:
the method further comprises, for each combination of the potential combinations, predicting one or more additional properties or results of the units of the combination device when formed from the first and second components of the combination, using one or more additional predictive models; and
the objective function includes a plurality of function terms each corresponding to a different property or result of the combination device.

8. The method of claim 1, wherein:
the combination device is a filled syringe;
the first component is a syringe; and
the second component is a fluid drug product.

9. The method of claim 8, wherein the one or more characteristics of the first component include one or more of glide force, shield removal force, barrel diameter, crush test results, plunger diameter, or plunger weight, and wherein the one or more characteristics of the second component include one or more of viscosity, density, or protein concentration.

10. (canceled)

11. The method of claim 1, wherein:
the combination device is an autoinjector;
the first component is a pre-filled syringe; and
the second component is an autoinjector subassembly.

12. The method of claim 11, wherein the one or more characteristics of the first component include one or more of extrusion force, breakloose force, protein concentration, or particle characterization, and wherein the one or more characteristics of the second component include one or more of specification number, saline release testing results, injection time, activation force, or spring force.

13. (canceled)

14. The method of claim 1, wherein predicting the property or result of the units of the combination device includes at least one of: (a) predicting a value indicative of variability in injection time or activation force across the units of the combination device, (b) predicting a probability distribution of a characteristic of the combination device across the units of the combination device, or (c) predicting a presence, amount, frequency, or likelihood of user complaints associated with the units of the combination device.

15. (canceled)

16. (canceled)

17. (canceled)

18. (canceled)

19. (canceled)

20. (canceled)

21. One or more non-transitory, computer-readable media storing instructions that, when executed by one or more processors, cause the one or more processors to:
identify a plurality of potential combinations of at least first components and second components, wherein each combination of the potential combinations can form one or more units of a combination device;
for each combination of the potential combinations, predict a property or result of the units of the combination device when formed from at least the first and second components of the combination, at least by applying (i) values of one or more characteristics of the first component of the combination, and (ii) values of one or more characteristics of the second component of the combination, as inputs to a predictive model;
select, based on the predicted properties or results for the potential combinations, a subset of combinations from among the potential combinations; and
provide an indication of the selected subset of combinations.

22. The one or more non-transitory, computer-readable media of claim 21, wherein:
each combination of the potential combinations is a different pair consisting of (i) a batch of the first components and (ii) a batch of the second components; and
for each combination of the potential combinations,
the respective values of the one or more characteristics of the first component are statistically representative of the respective batch of the first components, and
the respective values of the one or more characteristics of the second component are statistically representative of the respective batch of the second components.

23. The one or more non-transitory, computer-readable media of claim 22, wherein identifying the plurality of potential combinations includes omitting infeasible combinations.

24. The one or more non-transitory, computer-readable media of claim 21, wherein selecting the subset of combinations includes solving an objective function using the predicted properties or results for the potential combinations as inputs to the objective function.

25. The one or more non-transitory, computer-readable media of claim 21, wherein:
the combination device is a filled syringe;
the first component is a syringe; and
the second component is a fluid drug product.

26. The one or more non-transitory, computer-readable media of claim 25, wherein the one or more characteristics of the first component include one or more of glide force, shield removal force, barrel diameter, crush test results, plunger diameter, or plunger weight, and wherein the one or more characteristics of the second component include one or more of viscosity, density, or protein concentration.

27. (canceled)

28. The one or more non-transitory, computer-readable media of claim 21, wherein:
the combination device is an autoinjector;
the first component is a pre-filled syringe; and
the second component is an autoinjector subassembly.

29. The one or more non-transitory, computer-readable media of claim 28, wherein the one or more characteristics of the first component include one or more of extrusion force, breakloose force, protein concentration, or particle characterization, and wherein the one or more characteristics of the second component include one or more of specification number, saline release testing results, injection time, activation force, or spring force.

30. (canceled)

31. The one or more non-transitory, computer-readable media of claim 21, wherein predicting the property or result of the units of the combination device includes at least one of: (a) predicting a value indicative of variability in injection time or activation force across the units of the combination device, (b) predicting a probability distribution of a characteristic of the combination device across the units of the combination device, or (c) predicting a presence, amount, frequency, or likelihood of user complaints associated with the units of the combination device.

32. (canceled)

33. (canceled)

34. (canceled)

35. (canceled)
